(19) United States
(12) Patent Application Publication
(10) Pub. No.: US 2023/0398056 A1
(43) Pub. Date: Dec. 14, 2023

(54) RESIN COMPOSITION, GLUE DRIPPING MATERIAL, GLUE PRESSING MATERIAL, PREPARATION METHODS THEREFOR AND USES THEREOF

(71) Applicant: Yong CHEN, Guangzhou City (CN)

(72) Inventor: Yong CHEN, Guangzhou City (CN)

(21) Appl. No.: 18/250,407

(22) PCT Filed: Apr. 9, 2021
(86) PCT No.: PCT/CN2021/086206

§ 371 Date: Apr. 25, 2023

(30) Foreign Application Priority Data
Mar. 4, 2021 (CN) 202110239966.0

(51) Int. Cl. A61K 8/87; A61Q 3/00; C09J 175/16

(52) U.S. Cl. CPC A61K 8/87; A61Q 3/00; C09J 175/16

(57) ABSTRACT

The present disclosure provides a resin composition, an adhesive-dropping material, an adhesive-pressing material, and a preparation method and application thereof. The resin composition comprises the following components: an ester-based prepolymer; a reactive diluent; and a photoinitiator; wherein the addition amount of the ester-based prepolymer is 60 to 80 parts by weight, the addition amount of the reactive diluent is 15 to 30 parts by weight, and the addition amount of the photoinitiator is 1 to 5 parts by weight, based on the part by weight. The artificial nail prepared by using the resin composition, the adhesive-dropping material, and the adhesive-pressing material according to the present disclosure has high mimicry. When in use, the artificial nail which is not completely hardened may be attached to the surface of the real nail and then hardened by UV light, so that the artificial nail is more conformable.

### TECHNICAL FIELD

The present disclosure relates to a resin composition, an adhesive-dropping material, an adhesive-pressing material, and a preparation method and application thereof, in particular to a resin composition, an adhesive-dropping material, an adhesive-pressing material, and a preparation method thereof, and an unhardened artificial nail. The present disclosure belongs to the field of artificial nails.

### BACKGROUND

With the popularity of nail art such as decorating small artificial gemstones, drawing patterns, pasting ornaments, and adhering artificial nails on nails (fingernails), nail cosmetic materials and nail art technology using nail cosmetic materials have developed rapidly. Conventional nail cosmetic materials typically are nail polish and glaze composed of nitrocellulose, alkyd resin, and solvents such as ethyl acetate or butyl acetate. The coating film is formed by laminating and coating three layers of the above materials on the nail surface in the order of base coat (bottom layer), colored nail polish (intermediate layer), and clear nail polish (top layer). However, due to the use of organic solvents, the drying time is long, and it is difficult to dry the inside and apply a thick coating. Furthermore, organic solvents such as acetone, ethyl acetate, and butyl acetate are used as stripping agents in the peeling operation (removal) of the coating film accompanying the growth of nails, so that health damage such as organic solvent poisoning may be caused by direct inhalation and skin absorption of users.

In recent years, nail cosmetic materials using a photocurable resin composition called gel nails have attracted much attention because they do not contain organic solvents, may be cured in a short time, and are excellent in applying a thick coating and glazing property. Gel nails are mainly composed of polymers or oligomers, monomers, and photopolymerization initiators, which are coated on the surface of nails and irradiated by an ultraviolet (UV) exposure machine or light emitting diode (LED) exposure machine as a light source to form a cured coating film. However, the edge of the conventional gel nail is vertically cut, which is easy to scratch with foreign objects when in use, and the nail effect is unreal.

Patent document 1 discloses a gel nail sticker and a method of manufacturing the same. The gel nail sticker comprises: a lower laminated part which is a part directly attached to a nail or a toenail, and has a color or a pattern, and is formed of a flexible material to correspond to a curved surface of the nail or the toenail; and an upper laminated part which is a transparent coated layer positioned on the lower laminated part and providing glossiness to a color or a pattern of the lower laminated part, and is in a flexible semi-solid state to correspond to the curved surface of the nail or the toenail before being attached to the nail or the toenail, and is cured to a solid state while maintaining a form attached to the nail or the toenail when an ultraviolet ray is irradiated to the upper laminated part after the upper laminated part is attached to the nail or the toenail. The edge of this gel nail sticker is vertically cut, which is easy to scratch with foreign objects when in use.

Patent document 2 discloses a photocurable nail cosmetic material comprising a polyurethane (A) having a (meth) acrylamide group. Said material may form nail stickers or nail sheets by light curing. However, the edge of the cured nail sticker or nail sheet is still vertically cut, which is easy to scratch with foreign objects when in use.

### PATENT DOCUMENT

Patent document 1: CN 107690292A

Patent document 2: CN 110025504A

### SUMMARY

### Technical Problem

In view of the technical problems existing in the prior art, for example, the edge of the conventional gel nail is vertically cut, which is easy to scratch with foreign objects when in use, and the nail effect is unreal, etc. The present disclosure firstly provides a resin composition, adhesive-dropping material, and an adhesive-pressing material. The artificial nail prepared by using the resin composition, the adhesive-dropping material, and the adhesive-pressing material according to the present disclosure has high mimicry. When in use, the artificial nail which is not completely hardened may be attached to the surface of the real nail and then hardened by UV light, so that the artificial nail is more conformable.

Further, the present disclosure also provides an adhesive-dropping material, an adhesive-pressing material, and a preparation method of artificial nails. Both of the aforementioned methods are simple and easy to operate, and the raw materials are easy to obtain.

### Solution to the Problems

[1]. A resin composition, comprising the following components of:
an ester-based prepolymer;
a reactive diluent; and
a photoinitiator; wherein
the addition amount of the ester-based prepolymer is 60 to 80 parts by weight, the addition amount of the reactive diluent is 15 to 30 parts by weight, and the addition amount of the photoinitiator is 1 to 5 parts by weight, based on the part by weight.
[2]. The resin composition according to [1], wherein the ester-based prepolymer has a number-average molecular weight of 1000 to 20000 Da; preferably, the ester-based prepolymer comprises one or a combination of two or more of polyurethane, poly (meth) acrylate, epoxy (meth) acrylate, polyurethane (meth) acrylate, polyester (meth) acrylate, epoxy resin; more preferably, the ester-based prepolymer comprises poly (meth) acrylate and epoxy (meth) acrylate, further preferably, the weight ratio of the poly (meth) acrylate to the epoxy (meth) acrylate is 0.1 to 20:1.
[3]. The resin composition according to [2], wherein the polyurethane (meth) acrylate is obtained by reacting (i) a compound having two or more isocyanate groups in a molecule, (ii) a (meth) acrylate having at least one hydroxyl group in a molecule, and (iii) a polyol compound having two or more hydroxyl groups in a molecule.
[4]. The resin composition according to [2] or [3], wherein the epoxy (meth) acrylate is obtained by reacting (a) an epoxy compound having at least one epoxy group with (b) (meth) acrylic acid.
[5]. The resin composition according to any one of [1] to [4], wherein the resin composition further comprises a filler; preferably, the addition amount of the filler is 1 to 5 parts by weight.
[6]. An adhesive-pressing material, wherein the adhesive-pressing material comprises the resin composition according to any one of [1] to [5]; preferably, the adhesive-pressing material has a viscosity of 1000000 to 2000000 Pa's at 60° C., and the adhesive-pressing material has a viscosity of 500000 to 800000 Pas at 80° C.
[7]. A method for preparing the adhesive-pressing material according to [6], comprising a step of mixing each component of the resin composition.
[8]. An artificial nail, comprising:
an adhesive layer;
a resin layer in contact with the adhesive layer; and,
an adhesive-pressing layer formed on a surface of the resin layer; wherein
the adhesive-pressing layer is derived from the adhesive-pressing material according to [6].
[9]. The artificial nail according to [8], wherein the thickness of the adhesive-pressing layer close to the nail tip is less than or equal to the thickness of the adhesive-pressing layer close to the nail root.
[10]. The artificial nail according to [8] or [9], wherein the adhesive-pressing layer has a thickness of 0.01 to 3 mm; the resin layer has a thickness of 0.02 to 0.2 mm; and the adhesive layer has a thickness of 0.01 to 0.3 mm.
[11]. The artificial nail according to any one of [8] to [10], further comprising at least one of a pattern layer, a base layer, an ornament layer and a protective layer: wherein
the pattern layer is provided on a surface of the resin layer so that the adhesive-pressing layer is in contact with the pattern layer;
the base layer is peelably provided on the side of the adhesive layer opposite to the resin layer;
the ornament layer is provided on the side of the adhesive-pressing layer far away from the resin layer; and
the protective layer is provided between the adhesive-pressing layer and the ornament layer.
[12]. A method for preparing the artificial nail according to any one of [8] to 11, comprising a step of composite molding each layer structure of the artificial nail.
[13]. An adhesive-dropping material, wherein the adhesive-dropping material comprises the resin composition according to any one of [1] to [4]; preferably, the adhesive-dropping material has a viscosity of 1500 to 2000 Pa·s at 60° C., and the adhesive-dropping material has a viscosity of 500 to 800 Pa·s at 80° C.
[14]. A method for preparing the adhesive-dropping material according to [13], comprising a step of mixing each component of the resin composition.
[15]. An artificial nail, comprising:
an adhesive layer;
a resin layer in contact with the adhesive layer; and,
an adhesive-dropping layer formed on a surface of the resin layer: wherein
the adhesive-dropping layer is derived from the adhesive-dropping material according to [13]; and,
the adhesive-dropping material enables the surface of the adhesive-dropping layer to form a natural arc shape from the center to the edge of the adhesive-dropping layer.
[16]. The artificial nail according to [15], wherein the thickness of the adhesive-dropping layer close to the nail tip is less than or equal to the thickness of the adhesive-dropping layer close to the nail root.
[17]. The artificial nail according to [15] or [16], wherein the adhesive-dropping layer has a thickness of 0.01 to 3 mm; the resin layer has a thickness of 0.02 to 0.2 mm; and the adhesive layer has a thickness of 0.01 to 0.3 mm.
[18]. The artificial nail according to any one of [15] to [17], further comprising at least one of a pattern layer, a base layer, an ornament layer and a protective layer: wherein
the pattern layer is provided on a surface of the resin layer so that the adhesive-dropping layer is in contact with the pattern layer;
the base layer is peelably provided on the side of the adhesive layer opposite to the resin layer;
the ornament layer is provided on the side of the adhesive-dropping layer far away from the resin layer; and
the protective layer is provided between the adhesive-dropping layer and the ornament layer.
[19]. A method for preparing the artificial nail according to any one of [15] to [18], comprising a step of composite molding each layer structure of the artificial nail.

### The Effects

The artificial nail prepared by using the resin composition, the adhesive-dropping material, and the adhesive-pressing material according to the present disclosure has high mimicry. When in use, the artificial nail which is not completely hardened may be attached to the surface of the real nail and then hardened by UV light, so that the artificial nail is more conformable.

Further, the adhesive-dropping material, the adhesive-pressing material, and the artificial nail according to the present disclosure have the advantages of a simple production process, easily obtained raw materials and low cost, and are suitable for mass production.

### BRIEF DESCRIPTION OF THE DRAWINGS

FIG. 1 illustrates a cross-sectional structure of an adhesive artificial nail according to an embodiment of the present disclosure.

FIG. 2 illustrates a cross-sectional structure of a pressure-sensitive adhesive artificial nail according to an embodiment of the present disclosure, and the semi-solid photosensitive resin layer in FIG. 2 is the adhesive-pressing layer according to the present disclosure.

### DETAILED DESCRIPTION

Various exemplary embodiments, features, and aspects of the present disclosure will be described in detail below. As used herein, the term “exemplary” means “serving as an example, embodiment, or illustration.” Any embodiment described herein as “exemplary” is not necessarily to be construed as preferred or advantageous over other embodiments.

In addition, numerous specific details are set forth in the following detailed description in order to better explain the present disclosure. It should be understood by those skilled in the art that the present disclosure may be practiced without certain specific details. In other embodiments, methods, means, apparatus, and steps well known to those skilled in the art have not been described in detail in order to highlight the subject matter of the present disclosure.

In the present disclosure, the term “(meth) acrylate” used herein includes the meanings of “acrylate” as well as “methacrylate”; and the term “(meth) acrylic acid” used herein includes the meanings of “acrylic acid” as well as “methacrylic acid”.

Unless otherwise stated, the units used herein are international standard units, and the numerical values and numerical ranges appearing in the present disclosure should be understood to include systematic errors that are inevitable in industrial production.

In the present disclosure, unless otherwise specified, “%” refers to mass percentage.

In the present disclosure, the meaning expressed by “may” includes both the meanings of performing a certain treatment and the meaning of not performing a certain treatment.

In the present disclosure, references to “some specific/preferred embodiments”, “other specific/preferred embodiments”, “embodiments” and the like refer to a particular element (e.g., feature, structure, property, and/or characteristic) described in connection with the embodiment is included in at least one embodiment described herein, and may or may not be present in other embodiments. In addition, it should be understood that the elements may be combined in any suitable manner in the various embodiments.

In the present disclosure, the numerical range indicated by “numerical value A to numerical value B” refers to the range including the numerical values A and B of the end points.

<First Aspect>

A first aspect of the present disclosure provides a resin composition, comprising the following components of:
an ester-based prepolymer:
a reactive diluent; and
a photoinitiator; wherein
the addition amount of the ester-based prepolymer is 60 to 80 parts by weight, the addition amount of the reactive diluent is 15 to 30 parts by weight, and the addition amount of the photoinitiator is 1 to 5 parts by weight, based on the part by weight.

The resin composition according to the present disclosure may form an adhesive-dropping material or an adhesive-pressing material. When the artificial nail is prepared by using the adhesive-dropping material or adhesive-pressing material, the artificial nail may have a natural arc-shaped transition and smooth edges, thus avoiding the risk of scraping with foreign objects caused by vertically cutting the edges of nails. At the same time, the nails have a good flatness and mimicry effect, and are soft without losing toughness.

<Ester-Based Polymer>

In order to better realize the features of the present disclosure, said ester-based prepolymer has a number-average molecular weight of 1000 to 20000 Da, preferably of 1000 to 10000 Da, for example: 1500 to 9500 Da, 1800 to 900 0 Da, 2000 to 8000 Da, 300) to 7000 Da, 4000 to 6000 Da, 4500 to 5500 Da, etc. When the number-average molecular weight of the ester-based prepolymer according to the present disclosure is 1000 to 20000 Da, preferably 1000 to 10000 Da, the adhesive or pressure-sensitive adhesive may have a smoothly transition and maintain flatness when preparing the adhesive-dropping layer or adhesive-pressing layer of the artificial nail.

Specifically, in the present disclosure, said ester-based prepolymer includes one or a combination of two or more of polyurethane, poly (meth) acrylate, epoxy (meth) acrylate, polyurethane (meth) acrylate, polyester (meth) acrylate, epoxy resin.

Preferably, in the present disclosure, said ester-based prepolymer may be epoxy (meth) acrylate, and/or polyurethane (meth) acrylate, more preferably, the ester-based prepolymer according to the present disclosure includes epoxy (meth) acrylate, and polyurethane (meth) acrylate. Specifically, the weight ratio of the polyurethane (meth) acrylate to the epoxy (meth) acrylate is 0.1 to 20:1, preferably 0.5 to 10:1, more preferably 0.8 to 8:1, in order to maximize the efficacy of the ester-based prepolymer according to the present disclosure.

Polyurethane (Meth) Acrylate

The polyurethane (meth) acrylate may be synthesized from a reaction product of (i) a compound having two or more isocyanate groups in a molecule, (ii) a (meth) acrylate having at least one hydroxyl group in a molecule, and (iii) a polyol compound having two or more hydroxyl groups in a molecule.

Specifically, (i) the compound having two or more isocyanate groups in the molecule is not particularly limited, and examples thereof include aromatic polyisocyanates, alicyclic polyisocyanates, aliphatic polyisocynates, etc.

Aromatic polyisocyanates are not particularly limited, and examples thereof include 2,4-toluene diisocyanate, 2,6-toluene diisocyanate, 1,3-xylylene diisocyanate, 1,4-xylylene diisocyanate, tetramethyl xylylene diisocyanate, diphenylmethane diisocyanate, naphthalene-1,5-diisocyanate, triphenylmethane triisocyanate, hydrogenate phenylmethane diisocyanate, toluene diisocyanate, and the like.

Alicyclic polyisocyanates are not particularly limited, and examples thereof include hexamethylene diisocyanate, isophorone diisocyanate, bis (4-isocyanatocyclohexyl) methane, 1,3-bis (isocyanatomethyl) cyclohexane, 1,4-bis (isocyanatomethyl) cyclohexane, norbornane diisocyanate, bicycloheptane triisocyanate, and the like.

Aliphatic polyisocynates are not particularly limited, and examples thereof include hexamethylene diisocyanate, 1,3,6-hexamethylene triisocyanate, 1,6,11-undecane triisocyanate, and the like.

The aromatic polyisocyanate is preferably used in the present disclosure in consideration of smooth transition and maintenance of flatness at the time of adhesive dropping or pressing.

(ii) The (meth) acrylate having at least one hydroxyl group in the molecule is not particularly limited, and examples thereof include mono (meth) acrylates of dihydric alcohols such as ethylene glycol, propylene glycol, 1,3-propanediol, 1,3-butanediol, 1,4-butanediol and polyethylene glycol, etc; and mono (meth) acrylates or di (meth) acrylates of trihydric alcohols such as trimethylolethane, trimethylolpropane and glycerol, etc. Among them, a mono (meth) acrylate of a dihydric alcohol is preferred, and a mono (meth) acrylate of an ethylene glycol such as hydroxyethyl methacrylate is more preferred from the viewpoint of obtaining a cured product with excellent flexibility. These (meth) acrylates having one or more hydroxyl groups may be used alone or in combination.

Further, (iii) the polyol compound having two or more hydroxyl groups in the molecule is not particularly limited, and examples thereof include polyether polyol, polyester polyol, bisphenol polyol, polyisoprene polyol, hydrogenate polyisoprene polyol, polybutadiene polyol, hydrogenated polybutadiene polyol, polycarbonate polyol, and the like. Among them, polycarbonate polyol, polybutadiene polyol, and hydrogenated polybutadiene polyol are preferred from the viewpoint of excellent transparency and durability, and polycarbonate polyol is particularly preferred from the viewpoint of no clouding of the cured product in a high-temperature and high-humidity atmosphere. In the present disclosure, said polycarbonate polyol has a weight-average molecular weight of 800 to 2000 Da.

In one embodiment, the molar ratio of (iii) the polyol compound having two or more hydroxyl groups in the molecule, (1) the compound having two or more isocyanate groups in the molecule, and (ii) the (meth) acrylate having at least one hydroxyl group in the molecule is (1-3):(2-4):(1-2). When the molar ratio is within said range, more excellent technical effects may be realized.

In addition, the catalyst used in the synthesis is not particularly limited, and examples thereof include lead oleate, tetrabutyltin, antimony trichloride, triphenylaluminum, trioctylaluminum, dibutyltin dilaurate, copper naphthenate, zinc naphthenate, zinc octanoate, zinc octenate, zirconium naphthenate, cobalt naphthenate, tetrabutyl-1,3-diacetoxydistannoxane, triethylamine, 1,4-diaza [2,2,2]dicyclooctane, N-ethylmorpholine, and the like. Among them, one or a combination of two or more of dibutyltin dilaurate, zinc naphthenate, zinc octanoate, zinc octenate and the like is preferred from the viewpoint of obtaining a cured product with high activity and better transparency. These catalysts are preferably used in an amount of 0.01 to 10 wt % based on the total mass of 100 parts by mass of the reaction product, based on the total mass of the polyurethane (meth) acrylate. In addition, the reaction temperature is generally preferably 10 to 100° C., and particularly preferably 30 to 90° C.

Further, the reaction of the synthesized polyurethane (meth) acrylate is an exothermic reaction, and polymerization inhibitors need to be added to prevent other unwanted polymerization. Specifically, in the present disclosure, commonly used polymerization inhibitors are p-hydroxyphenylmethane, hydroquinone, 4-methoxyphenol, p-hydroxyanisole, 2,5-dimethylhydroquinone, 2,6-di-tert-butyl p-phenylcresol, etc. The addition amount is about 0.01 wt % to 0.1 wt %, preferably 0.03 to 0.08 wt %.

Epoxy (Meth) Acrylate

In the present disclosure, said epoxy (meth) acrylate may be obtained by reacting (a) an epoxy compound having at least one epoxy group with (b) (meth) acrylic acid.

Specifically, (a) the epoxy compound having at least one epoxy group is preferably an epoxy compound having two or more epoxy groups, and a bisphenol-type epoxy resin may be specifically used. In particular:

in some preferred embodiments according to the present disclosure, an epoxy compound suitable for the present disclosure has a structure represented by the following general formula (I):

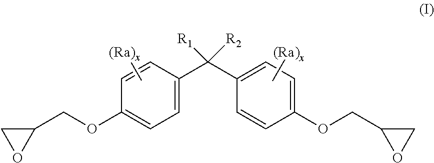

wherein, R₁ and R₂ are the same or different and each independently represents a hydrogen atom, an aryl group having 6 to 30 carbon atoms, or an alkyl group or alkoxy group having 1 to 10 carbon atoms, and preferably independently represents a hydrogen atom or an alkyl group having 1 to 4 carbon atoms;
R₃ is the same or different at each occurrence and is independently selected from the group consisting of hydrogen, halogen and an alkyl having 1 to 5 carbon atoms; preferably an alkyl group having 1 to 3 carbon atoms; x is an integer of 0 to 4, preferably an integer of 0 or 1. The halogen may be an F, Cl or Br atom.

Specifically, the epoxy compound suitable for the present disclosure has a structure represented by the following general formulas (I-1) to (I-3):

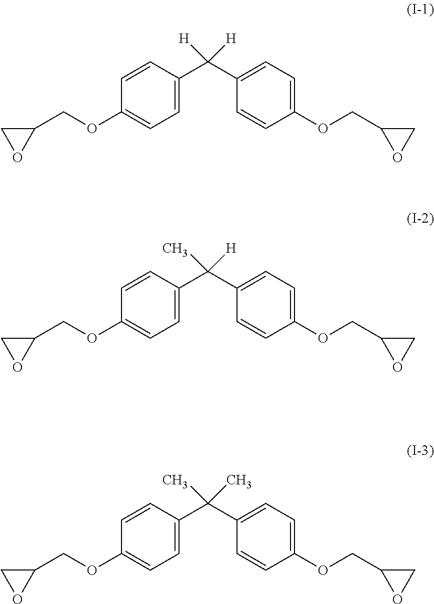

In other embodiments of the present disclosure, the epoxy compound suitable for the present disclosure has a structure represented by the following general formulas (II):

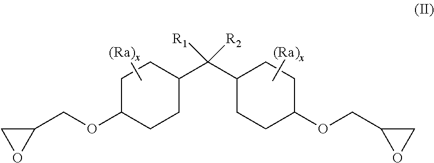

wherein, R₁ and R₂ are the same or different and each independently represents a hydrogen atom, an aryl group having 6 to 30 carbon atoms, or an alkyl group or alkoxy group having 1 to 10 carbon atoms, and preferably independently represents a hydrogen atom or an alkyl group having 1 to 4 carbon atoms;
each R₃ is the same or different and is independently selected from the group consisting of hydrogen, halogen and an alkyl having 1 to 5 carbon atoms, preferably an alkyl group having 1 to 3 carbon atoms; x is an integer of 0 to 4, preferably an integer of 0 or 1.

Specifically, the epoxy compound suitable for the present disclosure has a structure represented by the following general formulas (II-1) to (II-3):

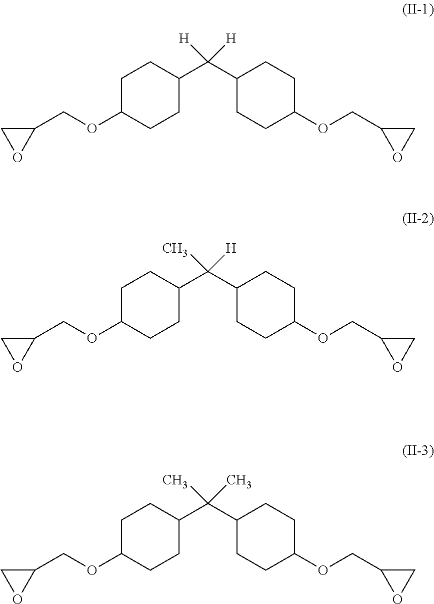

It should be noted that one epoxy compound or a mixture of two or more epoxy compounds may be used in the present disclosure, wherein the epoxy compound represented by formula (I-3) is a bisphenol A-type epoxy resin and the epoxy compound represented by formula (II-3) is a hydrogenated bisphenol A-type epoxy resin.

(b) The (meth) acrylic acid may be some (meth) acrylic acid commonly used in the art, such as acrylic acid, methacrylic acid, dimethacrylic acid and other commonly used acrylic acid.

In one specific embodiment, the molar ratio of the epoxy compound to methacrylic acid is 1:(0.8 to 1.2).

In addition, the catalyst used in the synthesis is not particularly limited, and tertiary amine, quaternary ammonium salt, triethylamine, N,N-dimethylbenzylamine, trimethylbenzylammonium chloride, triphenylphosphine, triphenylantimony, chromium acetylacetonate, tetraethylammonium bromide and the like may be used as the catalyst in the synthesis. These catalysts are preferably used in an amount of 0.1 to 0.5 wt % based on the total mass of 100 parts by mass of the reaction product, based on the total mass of the raw materials for synthesizing the polyurethane (meth) acrylate. In addition, the reaction temperature is generally preferably 50 to 200° C., and particularly preferably 60 to 150° C.

Further, the ring-opening esterification of (meth) acrylic acid and epoxy compounds is an exothermic reaction, and polymerization inhibitors need to be added to prevent the polymerization of (meth) acrylic acid and epoxy compounds. Specifically, in the present disclosure, commonly used polymerization inhibitors are p-hydroxyphenylmethane, hydroquinone, 4-methoxyphenol, 2,5-dimethylhydroquinone, 2,6-di-tert-butyl p-phenylcresol, etc. The addition amount is about 0.01 wt % to 0.1 wt %, preferably 0.03 to 0.08 wt %.

In some specific embodiments, for maximizing the effects of the present disclosure, the addition amount of the ester-based prepolymer is 60 to 80 parts by weight, for example, 62 parts by weight, 65 parts by weight, 68 parts by weight, 70 parts by weight, 72 parts by weight, 75 parts by weight, 78 parts by weight, etc., based on the part by weight.

<Reactive Diluent>

The composition according to the present disclosure further includes a reactive diluent. The reactive diluent according to the present disclosure is not particularly limited, and may be conventionally selected by those skilled in the art as required.

Examples of the reactive diluent include one or a combination of two or more of butyl (meth) acrylate, ethyl (meth) acrylate, methyl (meth) acrylate, glycidyl acrylate, glycidyl methacrylate, hydroxyethyl acrylate, hydroxyethyl methacrylate, isobornyl methacrylate, hydroxypropyl acrylate, hydroxypropyl methacrylate, N-vinylpyrinone, diethylene glycol phthalate diacrylate, isobornyl methacrylate, tripropylene glycol phthalate diacrylate, dipropylene glycol diacrylate, tripropylene glycol diacrylate, neopentyl glycol diacrylate, propoxylated neopentyl glycol diacrylate, 1,6-hexanediol diacrylate, 1,4-butanediol diacrylate, ethoxylated bisphenol A diacrylate, diethylene glycol diacrylate, triethylene glycol diacrylate, trimethylolpropane triacrylate, ethoxylated trimethylolpropane triacrylate, pentaerythritol triacrylate, propoxylated glycerol triacrylate, etc.

Preferably, the reactive diluent used in the present disclosure includes hydroxypropyl methacrylate and/or isobornyl methacrylate, and the like

In some specific embodiments, for maximizing the effects of the present disclosure, the addition amount of the reactive diluent is 15 to 30 parts by weight, for example, 18 parts by weight, 20 parts by weight, 22 parts by weight, 25 parts by weight, 28 parts by weight, etc., based on the part by weight.

<Photoinitiator>

The composition according to the present disclosure further includes a photoinitiator. The photoinitiator refers to a compound that decomposes and produces a free radical species, a cation species or an anion species by irradiating active energy rays such as ultraviolet rays or visible rays. The photoinitiator is not particularly limited, and the photoinitiator is a commonly used photoinitiator such as a radical-based photoinitiator.

Specifically, the free radical-based photoinitiator may include, but is not limited to, one or a combination of two or more of the following: 1-hydroxycyclohexylphenylketone, diethoxyacetophenone, 2-hydroxy-2-methyl-1-phenylpropane-1-one, benzil di methyl ketal, 4-(2-hydroxyethoxy) phenyl-(2-hydroxy-2-propyl) ketone, 1-hydroxycyclohexylphenylketone, 2-methyl-2-morpholino (4-thiomethylphenyl) propane-1-one, 2-benzyl-2-dimethylamino-1-(4-morpholinophenyl) butanone, 2-hydroxy-2-methyl-1-[4-(1-methylvinyl) phenyl] acetone oligomer, benzoin, benzoin methyl ether, benzoin ethyl ether, benzoin isopropyl ether, benzoin isobutyl ether, benzophenone, methyl o-benzoyl benzoate, 4-phenylbenzophenone, 4-benzoyl-4′-methyl-diphenyl sulfide, 3,3′,4,4′-tetra (tert-butyl carbonyl peroxide) benzophenone, 2,4,6-trimethylbenzophenone, 4-benzoyl-N,N-dimethyl-N-[2-(1-oxo-2-propenoxy)ethyl] benzyl ammonium bromide, (4-benzoylbenzyl) trimethyl ammonium chloride, 2-isopropylthioxanone, 4-isopropylthioxanone, 2,4-diethylthioxanone, 2,4-dichlorothioxanone, 1-chloro-4-propoxythioxanone, 2-(3-dimethylamino-2-hydroxy)-3,4-dimethyl-9H-thioxanone-9-one racemic chloride, 2,4,6-trimethylbenzoyl-diphenyl-phosphine oxide, bis(2,4,6-trimethylbenzoyl)-phenyl phosphine oxide, acyl phosphine oxide, etc.

In some specific embodiments, for maximizing the effects of the present disclosure, the addition amount of the photoinitiator is 1 to 5 parts by weight, for example, 2 parts by weight, 3 parts by weight, 4 parts by weight, etc., based on the part by weight.

<Other Components>

The composition according to the present disclosure further includes other components, and additives such as volatile solvents, fillers, silane coupling agents, plasticizers, defoamers, pigments, rust inhibitors, leveling agents, dispersants, rheology control agents, flame retardants, pigments and the like may be used in the present disclosure in addition to the above components without impairing the purpose of the present disclosure.

As for the volatile solvent, the volatile solvents according to the present disclosure is used for enhancing the fluidity of the adhesive-dropping layer or the adhesive-pressing layer, which is convenient for dropping the above components on the surface of the pattern layer (or the resin layer) or pressing the components on the surface of the pattern layer (or the resin layer) during processing to form the adhesive-dropping layer or the adhesive-pressing layer. Volatile solvents are not particularly limited in the present disclosure and may be the volatile solvents commonly used in the art. Specifically, the volatile solvents include volatile organic solvents such as hexafluoroisopropanol, tetrahydrofuran, dichloromethane, trichloromethane, propyl acetate, acetone and the like, preferably propyl acetate.

As for the filler, for the purpose of improving the elastic modulus, fluidity, and the like of a cured product, the resin composition according to the present disclosure may further contain a filler to such an extent that the storage stability is not impaired. As a specific example of such a filler, an inorganic powder (inorganic filler) may be used.

Examples of the filler (inorganic filler) for the inorganic powder include, but are not limited to, one or a combination of two or more of fumed silica, alumina, mica, ceramics, calcium carbonate, aluminum nitride, carbon powder, kaolin clay, dry clay mineral, dry diatomaceous earth, kaolin, and the like, and preferably fumed silica. These may be used alone, or two or more may be used in combination. Preferably, in the present disclosure, the filler is added in an amount of 1 to 5 parts by weight.

<Second Aspect>

A second aspect of the present disclosure provides an adhesive-dropping material, wherein the adhesive-dropping material comprises the resin composition according to the first aspect of the present disclosure; preferably, the adhesive-dropping material has a viscosity of 1500 to 2000 Pa·s at 60° C., and the adhesive-dropping material has a viscosity of 500 to 800 Pa·s at 80° C.

Further, the present disclosure further provides a method for preparing the adhesive-dropping material according to the present disclosure comprising a step of mixing the respective components of the resin composition. Further, the solid content of the adhesive-dropping material according to the present disclosure is 75 to 90 wt % when the adhesive-dropping material is treated at 160° C. for 3 h.

When the artificial nail is prepared by using the adhesive-dropping material according to the present disclosure, the artificial nail may have a natural arc-shaped transition and smooth edges, thus avoiding the risk of scraping with foreign objects caused by vertically cutting the edges of nails. At the same time, the nails have a good flatness and mimicry effect, and are soft without losing toughness. When the adhesive-dropping material according to the present disclosure is used, the artificial nail which is not completely hardened may be attached to the surface of the real nail, and the adhesion between the artificial nail and the real nail is good after being irradiated by ultraviolet light.

Further, the second aspect of the present disclosure further provides an adhesive-pressing material, wherein the adhesive-pressing material comprises the resin composition according to the first aspect of the present disclosure; preferably, the adhesive-pressing material has a viscosity of 1000000 to 2000000 Pa·s at 60° C., and the adhesive-pressing material has a viscosity of 500000 to 800000 Pa·s at 80° C.

Further, the present disclosure further provides a method for preparing the adhesive-pressing material according to the present disclosure, comprising a step of mixing each component of the resin composition.

When the artificial nail is prepared by using the adhesive-pressing material according to the present disclosure, the artificial nail may have a natural arc-shaped transition and smooth edges, thus avoiding the risk of scraping with foreign objects caused by vertically cutting the edges of nails. At the same time, the nails have a good flatness and mimicry effect, and are soft without losing toughness. When the adhesive-pressing material according to the present disclosure is used, the artificial nail which is not completely hardened may be attached to the surface of the real nail, and the adhesion between the artificial nail and the real nail is good after being irradiated by ultraviolet light.

<Third Aspect>

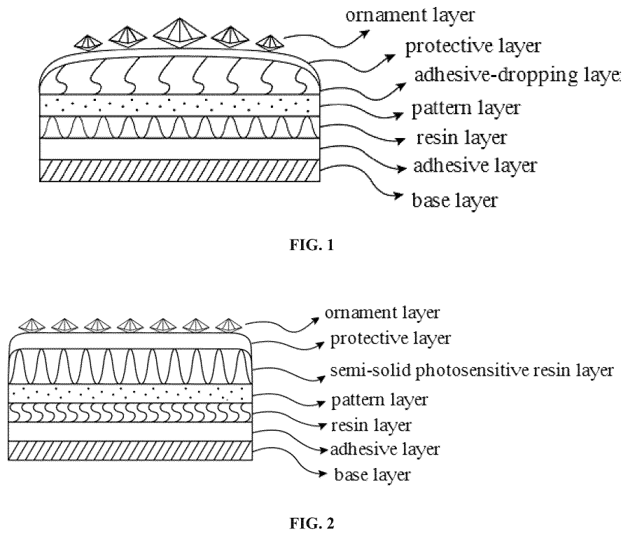

A third aspect of the present disclosure provides two types of artificial nails, that is, an artificial nail containing an adhesive-pressing layer (as shown in FIG. 2) and an artificial nail containing an adhesive-dropping layer (as shown in FIG. 1).

Specifically, an artificial nail containing an adhesive-dropping layer comprises:
an adhesive layer;
a resin layer in contact with the adhesive layer; and,
an adhesive-dropping layer formed on a surface of the resin layer; wherein
the adhesive-dropping layer is derived from the adhesive-dropping material according to the second aspect of the present disclosure; and,
the adhesive-dropping material enables the surface of the adhesive-dropping layer to form a natural arc shape from the center to the edge of the adhesive-dropping layer.

The artificial nail according to the present disclosure has high mimicry. When in use, the artificial nail which is not completely hardened may be attached to the surface of the real nail and then hardened by UV light, so that the artificial nail is more conformable.

Adhesive Layer

In the present disclosure, the purpose of the adhesive layer is to adhere the artificial nail to the nail. The raw material forming the adhesive layer in the present disclosure is not particularly limited and may be any feasible adhesive in the art.

In some specific embodiments, in the present disclosure, the adhesives may be hot-melt adhesives, room temperature-curing adhesives, and pressure sensitive adhesives; and pressure sensitive adhesives are preferred.

Specifically, the pressure-sensitive adhesive may be a rubber-type pressure-sensitive adhesive or a resin-type pressure-sensitive adhesive. For example, acrylate pressure-sensitive adhesives; synthetic rubber and regenerated rubber pressure-sensitive adhesives made of synthetic rubbers such as styrene-butadiene rubber, polyisobutylene, polyisoprene, butyl rubber, cis-butadiene rubber, chloroprene rubber, nitrile-butadiene rubber alone or in combination as the main body, combined with tackifying resin, softening agent, antioxidant, mixed solvent, etc; silicone and other resin-type pressure-sensitive adhesives, etc. In the present disclosure, the pressure-sensitive adhesive may be a silicone resin-type pressure-sensitive adhesive, and the pressure-sensitive adhesive PSA is preferably used as a raw material of the adhesive layer.

In addition, in the present disclosure, the average thickness of the adhesive layer may be 0.01 to 0.3 mm in consideration of the adhesion force to the nail and the degree of adhesion.

Resin Layer

The resin layer according to the present disclosure is in contact with the adhesive layer, and the raw material forming the resin layer in the present disclosure is not particularly limited and may be any feasible resin layer in the art.

In some specific embodiments, the raw material for forming the resin layer may be polyester, polyurethane, polyurea, polyether, polyorganosiloxane, perfluoropolyether, etc. In the present disclosure, polyurethane resin is preferably used as a raw material of the resin layer.

In some specific embodiments, a pattern layer may also be provided on the surface of the resin layer, thereby making the artificial nail more aesthetically pleasing. Specifically, the pattern layer may be provided with patterns by printing, gold stamping, spraying, printing, electroplating, transferring, and the like.

In addition, in the present disclosure, the average thickness of the resin layer is 0.02 to 0.2 mm.

Adhesive-Dropping Laver

The adhesive-dropping layer according to the present disclosure is formed on a surface of the resin layer, wherein the adhesive-dropping layer is derived from the adhesive-dropping material according to the second aspect of the present disclosure, and the adhesive-dropping material enables the surface of the adhesive-dropping layer to form a natural arc shape from the center to the edge of the adhesive-dropping layer. When the surface of the resin layer may also be provided with a pattern layer, the adhesive-dropping layer is in contact with the pattern layer.

The adhesive-dropping layer according to the present disclosure is derived from an adhesive-dropping material; when in use, the adhesive-dropping layer may be cured after being irradiated by an ultraviolet lamp.

Also, the adhesive-dropping material enables the surface of the adhesive-dropping layer to form a natural arc shape from the center to the edge of the adhesive-dropping layer. The adhesive-dropping material used in the present disclosure enables the surface of the adhesive-dropping layer to form a natural arc-shaped transition from the center to the edge of the adhesive-dropping layer (due to surface tension), so that hands may be prevented from being scraped.

In some specific embodiments, the thickness of the adhesive-dropping layer close to the nail tip is less than or equal to the thickness of the adhesive-dropping layer close to the nail root, which may be a configuration in which the artificial nail fits more closely to the human nail.

In addition, the average thickness of the adhesive-dropping layer is 0.01 to 3 mm. When the thickness of the adhesive-dropping layer is 0.01 to 3 mm, the heat release of artificial nails is smaller.

Other Layers

In the present disclosure, said artificial nail further comprises at least one of a pattern layer, a base layer, an ornament layer and a protective layer; wherein
the pattern layer is provided on a surface of the resin layer so that the adhesive-dropping layer is in contact with the pattern layer;
the base layer is peelably provided on the side of the adhesive layer opposite to the resin layer;
the ornament layer is provided on the side of the adhesive-dropping layer far away from the resin layer; and
the protective layer is provided between the ornament layer and the adhesive-dropping layer.

As for the pattern layer, the present disclosure does not specifically limit the specific material of the pattern layer, and may be some patterns commonly used in the art, thereby enabling the artificial nail to be more aesthetically pleasing.

As for the base layer, it may be a paper or non-woven structure (coated with release oil) of polyester (PET), polyethylene (PE), polypropylene (PP), ethylene-vinyl acetate copolymer (EVA), polyethylene octene co-elastomer (POE). Due to the existence of the base layer, the adhesive-dropping layer will not adhere to foreign matters before the artificial nail is used, and may better adhere to the nail.

As for the ornamental layer, the ornamental layer may be formed by adding artificial diamonds or other ornaments on the surface of the adhesive-dropping layer, thus enhancing the ornamental value of the structure.

As for the protective layer, resin, polyurethane, polyester, PET, and the like may be used. The protective layer is used to improve the brightness and wear resistance of products.

In addition, the length of the artificial nail according to the present disclosure may be longer from the length of the human nail, that is, when the artificial nail is attached to the nail, the artificial nail may be extended out. Ordinary nail stickers are flexible stickers, which are cut off at the edge of real nails when used because there is no support for the part extending out of real nails. However, due to the existence of ultraviolet curing resin, the artificial nail according to the present disclosure will be hardened after illumination, so that the artificial nail may extend out of the edge of a real nail.

The artificial nail containing an adhesive-pressing layer is basically the same as the artificial nail containing an adhesive-dropping layer except the adhesive-pressing layer.

Specifically, an artificial nail containing an adhesive-pressing layer comprises:
an adhesive layer;
a resin layer in contact with the adhesive layer; and,
an adhesive-pressing layer formed on a surface of the resin layer; wherein
the adhesive-pressing layer is derived from the adhesive-pressing material according to the second aspect of the present disclosure.

Adhesive-Pressing Laver

The adhesive-pressing layer according to the present disclosure is formed on a surface of the resin layer, wherein the adhesive-pressing layer is derived from the adhesive-pressing material according to the second aspect of the present disclosure. When the surface of the resin layer may also be provided with a pattern layer, the adhesive-pressing layer is in contact with the pattern layer.

The adhesive-pressing layer according to the present disclosure is derived from an adhesive-pressing material: when in use, the adhesive-pressing layer may be cured after being irradiated by an ultraviolet lamp.

Moreover, after the adhesive-pressing material is dried, the edge of the adhesive-pressing layer may form a natural arc shape (based on shrinkage after drying), so that hands can be prevented from being scraped.

In some specific embodiments, the thickness of the adhesive-pressing layer close to the nail tip is less than or equal to the thickness of the adhesive-pressing layer close to the nail root, which may be a configuration in which the artificial nail fits more closely to the human nail.

In addition, the thickness of the adhesive-pressing layer is 0.01 to 3 mm. When the thickness of the adhesive-pressing layer is 0.01 to 3 mm, the heat release of artificial nails is smaller.

<Fourth Aspect>

A fourth aspect of the present disclosure provides a method for preparing the artificial nail according to the third aspect of the present disclosure, comprising a step of composite molding each layer structure of the artificial nail.

Specifically, the method for preparing an artificial nail containing an adhesive-dropping layer comprises the following steps of:
forming an adhesive layer;
coating resin on the surface of the adhesive layer to form a resin layer; and
dropping an adhesive on the surface of the resin layer by using an adhesive-dropping material to form an adhesive-dropping layer.

In some specific embodiments, after the resin layer is formed, the method further comprises a step of forming a pattern layer on the surface of the resin layer, and then performing adhesive-dripping on the surface of the pattern layer to form an adhesive-dropping layer.

In addition, the method for preparing an artificial nail further comprises the following steps of:
forming a resin layer;
coating an adhesive on the surface of the adhesive layer to form an adhesive layer; and
dropping an adhesive on the other surface of the resin layer by using an adhesive-dropping material to form an adhesive-dropping layer.

The pattern layer may be formed on the surface of the resin layer by at least one of printing, gold stamping, spraying, printing, electroplating, transferring, etc.

In some specific embodiments, the adhesive layer according to the present disclosure is formed on a base layer, and the adhesive layer may be peeled off from the base layer to form a resin layer on the other side of the adhesive layer.

In other specific embodiments, the method further comprises pasting an ornament layer on the surface of the adhesive-dropping layer; and/or forming a protective layer on the surface of the adhesive-dropping layer.

Specifically, the method for preparing an artificial nail containing an adhesive-pressing layer comprises the following steps of:
forming an adhesive layer;
coating resin on the surface of the adhesive layer to form a resin layer; and
coating an adhesive on the surface of the resin layer by using an adhesive-pressing material to form an adhesive-pressing layer.

In some specific embodiments, after the resin layer is formed, the method further comprises a step of forming a pattern layer on the surface of the resin layer, and then coating an adhesive on the surface of the pattern layer to form an adhesive-pressing layer.

In addition, the method for preparing an artificial nail further comprises the following steps of:
forming a resin layer;
coating an adhesive on the surface of the adhesive layer to form an adhesive layer; and;
performing adhesive pressing on the other surface of the resin layer by using an adhesive-pressing material to form an adhesive-pressing layer.

The pattern layer may be formed on the surface of the resin layer by at least one of printing, gold stamping, spraying, printing, electroplating, transferring, etc.

In some specific embodiments, the adhesive layer according to the present disclosure is formed on a base layer, and the adhesive layer may be peeled off from the base layer to form a resin laver on the other side of the adhesive layer.

In other specific embodiments, the method further comprises pasting an ornament layer on the surface of the adhesive-pressing layer; and/or forming a protective layer on the surface of the adhesive-pressing layer.

### EXAMPLES

Embodiments of the present disclosure will be described in detail below in connection with examples but those skilled in the art will understand that the following examples are intended to illustrate the present disclosure only and should not be taken as limiting the scope of the present disclosure. If specific conditions are not specified in the example, the conventional conditions or the conditions recommended by the manufacturer shall be followed. If the manufacturer is not indicated, the reagents or instruments used are conventional products that are commercial available.

### Example 1

Example 1 of the present disclosure provided a resin composition consisting of 60 parts of an ester-based prepolymer, 15 parts of a reactive diluent, 1 part of a photoinitiator, and 1 part of a filler, based on the part by weight.

The ester-based prepolymer was polyurethane (meth) acrylate and epoxy (meth) acrylate in a weight ratio of 1:1.

The monomers in the raw materials for preparing the polyurethane (meth) acrylate were hydrogenated phenylmethane diisocyanate, hydroxyethyl methacrylate, and polycarbonate polyol: wherein the weight-average molecular weight of polycarbonate polyol was 800 to 2000 Da, which was purchased from Shanghai Huihai Chemical Technology Co., Ltd.

The method for preparing the polyurethane (meth) acrylate was as follows:
(1) Polyester carbonate polyol, hydrogenated phenylmethane diisocyanate, and dibutyltin dilaurate were mixed and reacted at 45° C. until the isocyanate group in hydrogenated phenylmethane diisocyanate reached the theoretical value (the reaction was complete);
(2) hydroxyethyl methacrylate and p-hydroxyanisole were added and reacted at 65° C. until the isocyanate group was less than 0.15% to obtain polyurethane (meth) acrylate:
wherein, the molar ratio of the polyester carbonate polyol, the hydrogenated phenylmethane diisocyanate, and the hydroxyethyl methacrylate was 1:2:1.

The amount of dibutyltin dilaurate was 0.02 wt % of the total amount of raw materials for preparing polyurethane (meth) acrylate; and the amount of p-hydroxyanisole was 0.02 wt % of the total amount of raw materials for preparing polyurethane (meth) acrylate.

The epoxy (meth) acrylate was a hydrogenated bisphenol A-type epoxy acrylate. The monomer in the raw materials for preparing the hydrogenated bisphenol A-type acrylate was the compound represented by formula (II-3), methacrylic acid; and the compound represented by formula (II-3) had an epoxy equivalent of 220 to 240 g/eq and was purchased from Suzhou Senfeida Chemical Co., Ltd. with the product name SF-3000.

The preparation method of the hydrogenated bisphenol A-type epoxy acrylate comprised the following steps of: mixing the compound represented by formula (II-3), methacrylic acid, 4-methoxyphenol and triethylamine, and reacting at 95° C. until the acid value was lower than 5 mg KOH/g to obtain the hydrogenated bisphenol A-type epoxy acrylate.

The molar ratio of the compound represented by formula (II-3) to methacrylic acid was 1:0.8; the triethylamine accounted for 0.1 wt % of the total amount of raw materials for preparing the hydrogenated bisphenol A-type epoxy acrylate; and the 4-methoxyphenol accounted for 0.01 wt % of the total amount of raw materials for preparing the hydrogenated bisphenol A-type epoxy acrylate.

The reactive diluent was hydroxypropyl methacrylate, and the filler was fumed silica, which was purchased from Changtaiweina Chemical Plant in Shouguang, Shandong Province, and the model was CT-617; and the photoinitiator was 1-hydroxycyclohexyl phenyl ketone.

The method for preparing the adhesive/adhesive-pressing material comprised a step of mixing the ester-based prepolymer, the reactive diluent, the photoinitiator, and the filler to obtain the adhesive/adhesive-pressing material.

The artificial nail was prepared by using the above adhesive/adhesive-pressing material, and the artificial nail was sequentially composed of an ornament layer, a protective layer, an adhesive-dropping layer, a pattern layer, a resin layer, an adhesive layer, and a base layer.

The raw material for preparing the adhesive/adhesive-pressing layer was adhesive/adhesive-pressing material.

The adhesive/adhesive-pressing layer had a thickness of 2 mm, the resin layer had a thickness of 0.1 mm, and the adhesive layer had a thickness of 0.2 mm.

The preparation process of the artificial nail containing the adhesive-dropping layer comprised the following steps of:
(1) coating an adhesive layer raw material on a base layer to obtain an adhesive layer; (2) coating a resin layer raw material on the adhesive layer to form a resin layer; (3) forming a pattern layer on the resin layer by printing; (4) die-cutting after the die-cutting knife presets its shape: (5) dropping an adhesive, forming an adhesive-dropping layer on the surface of the pattern layer; (6) drying; (7) coating a protective layer on the adhesive-dropping layer to obtain a protective layer; (8) pasting ornaments to obtain an ornament layer; (9) covering the surface of the ornament layer with a protective film; (10) cutting the protective film; (11) putting into a light-proof bag to obtain.

The preparation process of the artificial nail containing the adhesive-pressing layer comprised the following steps of:
(1) coating an adhesive layer raw material on a base layer to obtain an adhesive layer; (2) coating a resin layer raw material on the adhesive layer to form a resin layer; (3) forming a pattern layer on the resin layer by printing; (4) die-cutting after the die-cutting knife presets its shape: (5) pressing an adhesive, forming an adhesive-pressing layer on the surface of the pattern layer; (6) drying, so that the edge of the adhesive-pressing layer shrinks to form an arc shape; (7) coating a protective layer on the adhesive-pressing layer to obtain a protective layer; (8) pasting ornaments to obtain an ornament layer; (9) covering the surface of the ornament layer with a protective film; (10) cutting the protective film: (11) putting into a light-proof bag to obtain.

### Example 2

Example 2 of the present disclosure provided a resin composition consisting of 80 parts of an ester-based prepolymer, 30 parts of a reactive diluent, 5 parts of a photoinitiator, and 5 parts of a filler, based on the part by weight.

The prepolymer was polyurethane (meth) acrylate and epoxy (meth) acrylate in a weight ratio of 5:1.

The monomers in the raw materials for preparing the polyurethane (meth) acrylate were hydrogenated phenylmethane diisocyanate, hydroxyethyl methacrylate, and polycarbonate polyol: wherein the weight-average molecular weight of polycarbonate polyol was 800 to 2000 Da, which was purchased from Shanghai Huihai Chemical Technology Co., Ltd.

The method for preparing the polyurethane (meth) acrylate was as follows:
(1) Polyester carbonate polyol, hydrogenated phenylmethane diisocyanate, and dibutyltin dilaurate were mixed and reacted at 45° C. until the isocyanate group in hydrogenated phenylmethane diisocyanate reached the theoretical value (the reaction was complete);
(2) hydroxyethyl methacrylate and p-hydroxyanisole were added and reacted at 65° C. until the isocyanate group was less than 0.15% to obtain polyurethane (meth) acrylate;
wherein, the molar ratio of the polyester carbonate polyol, the hydrogenated phenylmethane diisocyanate, and the hydroxyethyl methacrylate was 3:4:2.

The amount of dibutyltin dilaurate was 0.5 wt % of the total amount of raw materials for preparing polyurethane (meth) acrylate; and the amount of p-hydroxyanisole was 0.5 wt % of the total amount of raw materials for preparing polyurethane (meth) acrylate.

The epoxy (meth) acrylate was a hydrogenated bisphenol A-type epoxy acrylate. The monomer in the raw materials for preparing the hydrogenated bisphenol A-type acrylate was the compound represented by formula (II-3), methacrylic acid; and the compound represented by formula (II-3) had an epoxy equivalent of 220 to 240 g/eq and was purchased from Suzhou Senfeida Chemical Co., Ltd. with the product name SF-3000.

The preparation method of the hydrogenated bisphenol A-type epoxy acrylate comprised the following steps of: mixing the compound represented by formula (II-3), methacrylic acid, 4-methoxyphenol and triethylamine, and reacting at 95° C. until the acid value was lower than 5 mg KOH/g to obtain the hydrogenated bisphenol A-type epoxy acrylate.

The molar ratio of the compound represented by formula (II-3) to methacrylic acid was 1:1.2; the triethylamine accounted for 0.5 wt % of the total amount of raw materials for preparing the hydrogenated bisphenol A-type epoxy acrylate; and the 4-methoxyphenol accounted for 0.1 wt % of the total amount of raw materials for preparing the hydrogenated bisphenol A-type epoxy acrylate.

The reactive diluent was hydroxypropyl methacrylate, and the filler was fumed silica, which was purchased from Changtaiweina Chemical Plant in Shouguang, Shandong Province, and the model was CT-617; and the photoinitiator was 1-hydroxycyclohexyl phenyl ketone.

The method for preparing the adhesive-dropping material comprised a step of mixing the ester-based prepolymer, the reactive diluent, the photoinitiator, and the filler to obtain the adhesive-dropping material.

The artificial nail was prepared by using the above adhesive-dropping material, and the artificial nail was sequentially composed of an ornament layer, an adhesive-dropping layer, a pattern layer, a resin layer, an adhesive layer, and a base layer.

The raw material for preparing the adhesive-dropping layer was adhesive-dropping material.

The adhesive-dropping layer had a thickness of 2 mm, the resin layer had a thickness of 0.1 mm, and the adhesive layer had a thickness of 0.2 mm.

The preparation process of the artificial nail comprised the following steps of:
(1) coating an adhesive layer raw materials on a base layer to obtain an adhesive layer; (2) bonding the base layer on the adhesive layer; (3) forming a pattern on a resin film to obtain a patter layer; (4) die-cutting after the die-cutting knife presets its shape; (5) dropping an adhesive, forming an adhesive-dropping layer on the surface of the pattern layer; (6) drying; (7) pasting ornaments to obtain an ornament layer; (8) covering the surface of the ornament layer with a protective film; (9) cutting the protective film; (10) putting into a light-proof bag to obtain.

### Example 3

Example 3 of the present disclosure provided a resin composition consisting of 72.5 parts of an ester-based prepolymer, 21.5 parts of a reactive diluent, 2.8 parts of a photoinitiator, and 4 parts of a filler, based on the part by weight.

The ester-based prepolymer was polyurethane (meth) acrylate and epoxy (meth) acrylate in a weight ratio of 2.4:1.

The monomers in the raw materials for preparing the polyurethane (meth) acrylate were hydrogenated phenylmethane diisocyanate, hydroxyethyl methacrylate, and polycarbonate polyol; wherein the weight-average molecular weight of polycarbonate polyol was 800 to 200 0 Da, which is purchased from Shanghai Huihai Chemical Technology Co., Ltd.

The method for preparing the polyurethane (meth) acrylate was as follows:
(1) Polyester carbonate polyol, hydrogenated phenylmethane diisocyanate, and dibutyltin dilaurate were mixed and reacted at 45° C. until the isocyanate group in hydrogenated phenylmethane diisocyanate reached the theoretical value (the reaction was complete);
(2) hydroxyethyl methacrylate and p-hydroxyanisole were added and reacted at 65° C. until the isocyanate group was less than 0.15% to obtain polyurethane (meth) acrylate:

The molar ratio of the polyester carbonate polyol, the hydrogenated phenylmethane diisocyanate, and the hydroxyethyl methacrylate was 2:3:1.5.

The amount of dibutyltin dilaurate was 0.1 wt % of the total amount of raw materials for preparing polyurethane (meth) acrylate; and the amount of p-hydroxyanisole was 0.1 wt % of the total amount of raw materials for preparing polyurethane (meth) acrylate.

The epoxy (meth) acrylate was a hydrogenated bisphenol A-type epoxy acrylate. The monomer in the raw materials for preparing the hydrogenated bisphenol A-type acrylate was the compound represented by formula (II-3), methacrylic acid; and the compound represented by formula (II-3) had an epoxy equivalent of 220 to 240 g/eq and was purchased from Suzhou Senfeida Chemical Co., Ltd. with the product name SF-3000.

The preparation method of the hydrogenated bisphenol A-type epoxy acrylate comprised the following steps of: mixing the compound represented by formula (II-3), methacrylic acid, 4-methoxyphenol and triethylamine, and reacting at 95° C. until the acid value was lower than 5 mg KOH/g to obtain the hydrogenated bisphenol A-type epoxy acrylate.

The molar ratio of the compound represented by formula (II-3) to methacrylic acid was 1:1; the triethylamine accounted for 0.35 wt % of the total amount of raw materials for preparing the hydrogenated bisphenol A-type epoxy acrylate; and the 4-methoxyphenol accounted for 0.05 wt % of the total amount of raw materials for preparing the hydrogenated bisphenol A-type epoxy acrylate.

The reactive diluent was hydroxypropyl methacrylate, and the filler was fumed silica, which was purchased from Changtaiweina Chemical Plant in Shouguang, Shandong Province, and the model was CT-617: and the photoinitiator was 1-hydroxycyclohexyl phenyl ketone.

The method for preparing the adhesive/adhesive-pressing material comprised a step of mixing the ester-based prepolymer, the reactive diluent, the photoinitiator, and the filler to obtain the adhesive/adhesive-pressing material.

The artificial nail was prepared by using the above adhesive/adhesive-pressing material, and the artificial nail was sequentially composed of an ornament layer, a protective layer, an adhesive-dropping layer, a pattern layer, a resin layer, an adhesive layer, and a base layer.

The raw material for preparing the adhesive/adhesive-pressing layer was adhesive/adhesive-pressing material.

The adhesive/adhesive-pressing layer had a thickness of 2 mm, the resin layer had a thickness of 0.1 mm, and the adhesive layer had a thickness of 0.2 mm.

The preparation process of the artificial nail containing the adhesive-dropping layer comprised the following steps of:
(1) coating an adhesive layer raw material on a base layer to obtain an adhesive layer; (2) coating a resin layer raw material on the adhesive layer to form a resin layer; (3) forming a pattern layer on the resin layer by printing; (4) die-cutting after the die-cutting knife presets its shape; (5) dropping an adhesive, forming an adhesive-dropping layer on the surface of the pattern layer; (6) drying; (7) coating a protective layer on the adhesive-dropping layer to obtain a protective layer; (8) pasting ornaments to obtain an ornament layer; (9) covering the surface of the ornament layer with a protective film; (10) cutting the protective film: (11) putting into a light-proof bag to obtain.

The preparation process of the artificial nail containing the adhesive-pressing layer comprised the following steps of:
(1) coating an adhesive layer raw materials on a base layer to obtain an adhesive layer; (2) coating a resin layer raw material on the adhesive layer to form a resin layer; (3) forming a pattern layer on the resin layer by printing; (4) die-cutting after the die-cutting knife presets its shape; (5) pressing an adhesive, forming an adhesive-pressing layer on the surface of the pattern layer: (6) drying, so that the edge of the adhesive-pressing layer shrinks to form an arc shape; (7) coating a protective layer on the adhesive-pressing layer to obtain a protective layer; (8) pasting ornaments to obtain an ornament layer; (9) covering the surface of the ornament layer with a protective film: (10) cutting the protective film; (11) putting into a light-proof bag to obtain.

### Example 4

Example 4 of the present disclosure provided a resin composition, the specific embodiment of which was the same as that of Example 3, except that the epoxy (meth) acrylate was a bisphenol A-type epoxy acrylate.

The specific embodiment of the method for preparing the polyurethane (meth) acrylate was the same as that of Example 3.

The specific embodiment of the method for preparing the epoxy (meth) acrylate was the same as that of Example 3, except that the compound represented by formula (II-3) was replaced by the compound represented by formula (I-3) (purchased from Luoyang Yixuan Industry and Trade Co., Ltd.).

The method for preparing the adhesive-dropping material was the same as that of Example 3.

The structure and preparation method of the artificial nail prepared by using the above adhesive-dropping material were the same as those of Example 3.

### Example 5

Example 5 of the present disclosure provided a resin composition, the specific embodiment of which was the same as that of Example 3, except that hydroxyethyl methacrylate in the monomer in the raw materials for preparing the polyurethane (meth) acrylate was replaced by hydroxypropyl acrylate.

The specific embodiment of the method for preparing the polyurethane (meth) acrylate was the same as that of Example 3, except that hydroxyethyl methacrylate was replaced by hydroxypropyl acrylate.

The specific embodiment of the method for preparing the epoxy (meth) acrylate was the same as that of Example 3.

The method for preparing the adhesive-dropping material was the same as that of Example 3.

The structure and preparation method of the artificial nail prepared by using the above adhesive-dropping material were the same as those of Example 3.

### Example 6

Example 6 of the present disclosure provided an adhesive, the specific embodiment of which was the same as that of Example 3, except that hydrogenated phenylmethane diisocyanate in the monomer in the raw material for preparing the polyurethane (meth) acrylate was replaced by toluene diisocyanate.

The specific embodiment of the method for preparing the polyurethane (meth) acrylate was the same as that of Example 3.

The specific embodiment of the method for preparing the epoxy (meth) acrylate was the same as that of Example 3.

The method for preparing the adhesive-dropping material was the same as that of Example 3.

The structure and preparation method of the artificial nail prepared by using the above adhesive-dropping material were the same as those of Example 3.

Performance Testing
1. Apparent evaluation: the flatness of the adhesive/adhesive-pressing layer in the artificial nails of Examples 1 to 6 was evaluated respectively. If there was no concave and convex visible to the naked eye, it was recorded as good flatness, otherwise it was recorded as poor flatness; and rub the edge of the adhesive-dropping layer with the same force by hand, if there was no obvious stabbing pain, it was recorded as good smoothness, otherwise it was recorded as poor smoothness. The results were shown in Table 1 below.
2. Mimicry effect evaluation: 100 female evaluators aged 25 to 35 were selected to evaluate the mimicry effect of the artificial nails of Examples 1 to 6, and the number of persons with good mimicry effect was recorded. The results were shown in Table 1 below.

TABLE 1

| | | | Mimicry effect |
|---|---|---|---|
| | Flatness | Smoothness | (person) |
| Example 1 | good | good | 92 |
| Example 2 | good | good | 95 |
| Example 3 | good | good | 95 |
| Example 4 | fair | fair | 83 |
| Example 5 | good | good | 90 |
| Example 6 | fair | fair | 80 |

As could be seen from Table 1 the artificial nails prepared using the resin composition according to the present disclosure had excellent properties.

The above-described examples of the present disclosure are merely illustrative for the purpose of clearly illustrating the present disclosure and are not intended to limit the embodiments of the present disclosure. Other variations or modifications in different forms may be made on the basis of the above description for those of ordinary skill in the art. There is no need and cannot be an exhaustive list of all embodiments here. Any modifications, equivalent substitutions, improvements, etc. made within the spirit and principles of the present disclosure should be included within the scope of protection of the claims of the present disclosure.

What is claimed is:

1. A resin composition, comprising the following components of:
an ester-based prepolymer,
a reactive diluent; and
a photoinitiator, wherein
the addition amount of the ester-based prepolymer is 60 to 80 parts by weight, the addition amount of the reactive diluent is 15 to 30 parts by weight, and the addition amount of the photoinitiator is 1 to 5 parts by weight, based on the part by weight.

2. The resin composition according to claim 1, wherein the ester-based prepolymer has a number-average molecular weight of 1000 to 20000 Da; the ester-based prepolymer comprises one or a combination of two or more of polyurethane, poly (meth) acrylate, epoxy (meth) acrylate, polyurethane (meth) acrylate, polyester (meth) acrylate, epoxy resin.

3. The resin composition according to claim 2, wherein the polyurethane (meth) acrylate is obtained by reacting (i) a compound having two or more isocyanate groups in a molecule, (ii) a (meth) acrylate having at least one hydroxyl group in a molecule, and (iii) a polyol compound having two or more hydroxyl groups in a molecule.

4. The resin composition according to claim 2, wherein the epoxy (meth) acrylate is obtained by reacting (a) an epoxy compound having at least one epoxy group with (b) (meth) acrylic acid.

5. The resin composition according to claim 1, wherein the resin composition further comprises a filler.

6. An adhesive-pressing material, wherein the adhesive-pressing material comprises the resin composition according to claim 1; the adhesive-pressing material has a viscosity of 1000000 to 2000000 Pa·s at 60° C., and the adhesive-pressing material has a viscosity of 500000 to 800000 Pa·s at 80° C.

7. A method for preparing the adhesive-pressing material according to claim 6, comprising a step of mixing each component of the resin composition.

8. An artificial nail, comprising:
an adhesive layer,
a resin layer in contact with the adhesive layer; and,
an adhesive-pressing layer formed on a surface of the resin layer; wherein
the adhesive-pressing layer is derived from the adhesive-pressing material according to claim 6.

9. The artificial nail according to claim 8, wherein the thickness of the adhesive-pressing layer close to the nail tip is less than or equal to the thickness of the adhesive-pressing layer close to the nail root.

10. The artificial nail according to claim 8, wherein the adhesive-pressing layer has a thickness of 0.01 to 3 mm; the resin layer has a thickness of 0.02 to 0.2 mm; and the adhesive layer has a thickness of 0.01 to 0.3 mm.

11. The artificial nail according to claim 8, further comprising at least one of a pattern layer, a base layer, an ornament layer and a protective layer; wherein
the pattern layer is provided on a surface of the resin layer so that the adhesive-pressing layer is in contact with the pattern layer;
the base layer is peelably provided on the side of the adhesive layer opposite to the resin layer,
the ornament layer is provided on the side of the adhesive-pressing layer far away from the resin layer; and
the protective layer is provided between the adhesive-pressing layer and the ornament layer.

12. A method for preparing the artificial nail according to claim 8, comprising a step of composite molding each layer structure of the artificial nail.

13. An adhesive-dropping material, wherein the adhesive-dropping material comprises the resin composition according to claim 1; the adhesive-dropping material has a viscosity of 1500 to 2000 Pa·s at 60° C., and the adhesive-dropping material has a viscosity of 500 to 800 Pa·s at 80° C.

14. A method for preparing the adhesive-dropping material according to claim 13, comprising a step of mixing each component of the resin composition.

15. An artificial nail, comprising:
an adhesive layer,
a resin layer in contact with the adhesive layer; and,
an adhesive-dropping layer formed on a surface of the resin layer, wherein
the adhesive-dropping layer is derived from the adhesive-dropping material according to claim 12; and,
the adhesive-dropping material enables the surface of the adhesive-dropping layer to form a natural arc shape from the center to the edge of the adhesive-dropping layer.

16. The artificial nail according to claim 15, wherein the thickness of the adhesive-dropping layer close to the nail tip is less than or equal to the thickness of the adhesive-dropping layer close to the nail root.

17. The artificial nail according to claim 15, wherein the adhesive-dropping layer has a thickness of 0.01 to 3 mm; the resin layer has a thickness of 0.02 to 0.2 mm; and the adhesive layer has a thickness of 0.01 to 0.3 mm.

18. The artificial nail according to claim 15,
further comprising at least one of a pattern layer, a base layer, an ornament layer and a protective layer, wherein
the pattern layer is provided on a surface of the resin layer so that the adhesive-dropping layer is in contact with the pattern layer;
the base layer is peelably provided on the side of the adhesive layer opposite to the resin layer,
the ornament layer is provided on the side of the adhesive-dropping layer far away from the resin layer; and
the protective layer is provided between the adhesive-dropping layer and the ornament layer.

19. A method for preparing the artificial nail according to claim 15, comprising a step of composite molding each layer structure of the artificial nail.
